(19) United States
(12) Patent Application Publication
(10) Pub. No.: US 2026/0272800 A1
(43) Pub. Date: Sep. 17, 2026

(54) METHOD FOR DEMI-PERMANENT HAIR COLORING

(71) Applicant: BEAUTY & BUSINESS S.p.A., Milano (IT)

(72) Inventors: Massimo Fabbi, Mozzo (BG) (IT); Katiuscia Grevalcuore, Bergamo (IT); Luca Carminati, Curno (BG) (IT)

(73) Assignee: BEAUTY & BUSINESS S.p.A., Milano (IT)

(21) Appl. No.: 19/560,702

(22) Filed: Mar. 9, 2026

(30) Foreign Application Priority Data
Mar. 14, 2025 (IT) 102025000005258

(51) Int. Cl. A61K 8/37; A61K 8/22; A61K 8/36; A61K 8/42; A61K 8/86; A61Q 5/06

(52) U.S. Cl. CPC A61K 8/375; A61K 8/22; A61K 8/361; A61K 8/42; A61K 8/86; A61Q 5/065; A61K 2800/30; A61K 2800/33; A61K 2800/4322

(57) ABSTRACT

The invention relates to a method for demi-permanent hair colouring and the relative compositions.

### STATE OF THE ART

This U.S. Utility Application claims priority to and the benefit of Italian Patent Application No. 102025000005258, filed Mar. 14, 2025, the content of which is incorporated herein by reference in its entirety.

About 33% of women over the age of 18 and more than 10% of men over the age of 40 in Europe and the United States dye their hair. (Chem Res Toxicol. 2022 Jun. 20; 35(6): 901-915).

Given the widespread use of cosmetic products for dyeing hair, the need has arisen to reduce the environmental impact thereof, as well as to improve the toxicological profile thereof for users.

The main methods for dyeing hair are the semi-permanent system and the permanent or demi-permanent oxidative system. The first consists of the use of direct dyes that are deposited on the hair surface.

The oxidative system is based on the reaction of so-called primary intermediates with couplers; both are practically colourless. In the presence of air or oxidants such as hydrogen peroxide, the primary dyes, typically aromatic primary amines with an additional hydroxyl or amino group, substituted or unsubstituted, in the para or ortho position, react with couplers (secondary dyes) such as resorcinol, m-aminophenol, m-phenylenediamine, or 1-naphthol.

Since the size of the dye molecules thus formed inside the cuticle is greater than the size of the starting primary intermediates and highly diffusible couplers, they remain trapped inside the hair and for this reason no significant discolouration occurs with subsequent washes or by the action of external agents. The penetration of these dyes into hair is favoured by the pH: the more alkaline it is, the greater the opening of the hair cuticles and the penetration of the colour and therefore its permanence within the fibre (The Chemistry Mechanism of Hair Dyes, Middle East Journal of Science (2022) 8(2):173-193).

Table 1 summarises the main types of colouring currently on the market.

TABLE 1
Hair dyes

| HAIR | SEMI- | DEMI- | DEMI- | |
|---|---|---|---|---|
| COLOR | PER- | PERMANENT | PERMANENT | PER- |
| TYPE | MANENT | ALKALINE | ACID | MANENT |
| Color | 2-3 Weeks | 6-8 Weeks | 6-8 Weeks | Permanent |
| longevity | | | | |
| properties | | | | |
| Lightening | NO | NO | NO | YES |
| effect | | | | |
| Dye pH | 3-5 | 8-10 | 6.5-10 | 9-11 |
| pH mixture | — | 7.5-8.5 | 6.0-7 | 8.5-10.5 |
| with | | | | |
| activator | | | | |

Demi-permanent colouration, having a pH of the mixture dye+activator from weakly alkaline to weakly acidic (pH 6-8.5), has the characteristic of not lightening the natural base of hair but of depositing tone-on-tone colour, resulting in less damage to hair.

Demi-permanent colourations at acidic pH respect hair fibre more than those at alkaline pH but their colour performance in terms of intensity and duration is penalised, often requiring the addition of direct dyes to compensate for the low intensity of the result. In this type of product, ethoxylated emulsifiers play a fundamental role as they act as penetration enhancers thanks to their polyethoxylated units (WO2012168207).

To achieve acidic demi-permanent colouring, special activators are usually used with a buffer system capable of lowering the pH of the hair dye composition, which is generally alkaline to favour the solubilization of the colourings, avoiding the risk of precipitation especially at cold temperatures (<0° C.).

Permanent and demi-permanent oxidative dyes can be in the form of a cream, gel, liquid or solid. Cream hair dyes are the most popular because they adhere perfectly to hair without slipping, favouring an adequate coverage of grey hair and a uniform colour result. Furthermore, the presence of fatty substances makes them nourishing/moisturising for hair. However, since cream dye must be applied to hair using a brush, good application skill is required. In recent years, therefore, the demand for liquid permanent and demi-permanent colourations has increased.

Permanent dyes in liquid form are highly sought after by the market for their ease, speed of application, and versatility. In fact, they can be applied with a brush or shaker, facilitating the uniform distribution of the product even on long, thick or curly hair. Liquid demi-permanent dyes are particularly useful for toning after bleaching, in order to improve unwanted yellow/orange reflections due to melanin residues. In this case, it is very useful for the mixture with the activator to be transparent so as to allow the hairdresser to visually evaluate the progression of colour development in order to rinse the product at the most appropriate time. In fact, the application time varies according to the porosity of the hair and the intensity of the result to be obtained.

Liquid dyes are particularly delicate formulas in terms of stability. It is necessary to use appropriate precautions to prevent the system from clouding, the precipitation of colourings or other salts present in the formula (antioxidants, reductants, chelators, etc.), separations or the system losing viscosity/stickiness in mixture with the activator making it impossible to apply.

Liquid dyes consist of a colouring composition and an activator both in liquid form (viscosity <7000 cps, 5×10 rmm), to be mixed together in certain ratios just before use.

The colourant composition is generally hydro-alcohol-based while the activator is water-based. In the colourant and/or activator composition the presence of water-soluble emulsifiers, usually ethoxylated (for example oleth-2, oleth-3, laureth-2, laureth-3, undeceth-3, deceth-3, steareth-20), and suspending/solubilizing (such as propylene glycol, propanediol, etc.) in order to avoid precipitations and deposits of colourants or salts on the bottom of the container, avoid separations, promote the transparency of the product and favour an adequate viscosity of the mixture.

The colourant composition or the activator usually includes a polymer, often an acrylate, to increase the stickiness/tackiness of the mixture so as to prevent the product from slipping down when applied to hair.

As a result of the absence of a nourishing/emollient fatty phase, liquid dyes often require the addition of silicones that form a film on hair, making it easily detangleable, combable, soft and shiny.

These three categories of ingredients (ethoxylated emulsifiers, acrylic polymers and silicones) unfortunately have a significant impact on the environment.

The worldwide production of ethoxylates amounts to several million tons per year, with a significant impact on the “carbon footprint”, ethylene oxide being produced from ethylene derived from fossil hydrocarbons. Alternative ethanol production from plant biomass cannot meet the huge market demand for ethoxylate.

Another environmental problem linked to the use of ethylene oxide is the presence of 1,4-dioxane, a reaction by-product classified as toxic according to the criteria of Annex XIII of REACH and as a category 2 carcinogen according to the CLP (EC Regulation n.1272/2008), highly diffusible in the aquatic environment and difficult to remove in water purification processes. For this reason, many regulatory bodies have imposed very strict limits for the presence of 1,4-dioxane in cosmetics.

Ethylene oxide is also used for the production of propylene glycols and their derivatives (PEGs) widely used in the cosmetic industry, including hair dyes, such as PEG-20 stearate, PEG-40-hydrogenated castor oil, PEG-12 dimethicone, PEG/PPG-4/12 dimethicone.

Acrylic polymers are poorly biodegradable and at risk of accumulation in the environment (Applied Microbiology and Biotechnology (2021) 105:991-1006).

Silicones do not degrade easily and consequently their use in personal care products raises environmental concerns (WO 2023129710). This is particularly true for ethoxylated silicones (e.g. PEG-12 dimethicone, PEG/PPG-4/12 dimethicone).

WO2025/012318 describes a colouration for hair free of ingredients known to cause skin problems, such as ethoxylated compounds, PEGs and their derivatives, and silicones, however this patent does not cover liquid dyes, in fact examples 1,2,12 and 13 contain about 16% of a blend of fatty acids and fatty alcohols (in particular cetearyl alcohol which is solid at room temperature) incompatible with a liquid consistency. These are also alkaline pH oxidative permanent dyes (they contain relevant percentages of ammonia and dimethylglucamine).

U.S. Ser. No. 11/813,349 describes permanent dyes in gel form without emulsifiers/ethoxylates thanks to the use of dehydro-xanthan gum, but in the examples they are still mixed with activators containing ethoxylated ingredients and the mixture is not transparent.

Completely eliminating ethoxylated ingredients from both the dye and the activator is a challenge that is not easy to solve.

In recent years, the use of polyglycerol esters with fatty acids as an alternative to ethoxylated emulsifiers and as alternative conditioners to silicones (e.g. polyglyceryl-3 betainate acetate, polyglyceryl-3 betainate citrate) has become established. These ingredients are characterized by a high solubility in water which results in excellent biocompatibility and biodegradability. They can, however, be used in a narrow pH range (pH 4-8) that makes them suitable for the formulation of skin care products (wo2024260330), hair care in emulsion form (WO2012168207) and semi-permanent colourations (WO 2018109154, JP 6736741) but not very useful in the field of permanent and demi-permanent dyes, especially in liquid form.

WO 2021161203 proposes the use of esters of polyglycerol phosphates that are stable at alkaline pH but does not describe the use in dyes in liquid form because the polyglycerol phosphates are not soluble in water and/or alcohol with consequent problems of stability and transparency.

A liquid demi-permanent colouration is commercially available at neutral pH (pH=7) comprising polyglycerol esters and free of acrylic polymers and silicones. However, ethoxylated ingredients are present in the activator. The dye+activator mixture is not transparent and the product is unstable at temperatures below 0° C., with the presence of precipitate.

There is therefore a need to develop a liquid demi-permanent dye characterised by the absence of ethoxylated emulsifiers, PEGs and their derivatives, acrylic polymers and silicones. Such composition must be storage stable from −10° C. to +50° C., transparent when mixed with an activator, also free of ethoxylated emulsifiers.

### DISCLOSURE OF THE INVENTION

It has been found that by combining esters of polyglycerols with oleic acid or with a mixture of oleic alcohol and cocamide-MIPA (amides of coconut fatty acids with [N-(2-hydroxypropyl)amine] or (mono)isopropanolamine) it is possible to formulate a liquid acidic demi-permanent dye without ethoxylated emulsifiers, without PEGs and their derivatives, without acrylic polymers and without silicones.

The invention therefore relates to a method for the demi-permanent colouring of hair which comprises the application on hair of a transparent mixture consisting of:
a) a first composition free of ethoxylated products and polyethylene glycols comprising oxidative dyes and a mixture of polyglyceryl esters with oleic acid or a mixture of oleyl alcohol and cocamide MIPA;
b) a second ethoxylate-free formulation comprising an activator.

The compositions a) and b) are mixed in a weight ratio of 1:1 to 1:2.

The composition comprising oxidative colourants and a mixture of polyglyceryl esters with oleic acid or polyglyceryl esters with oleic alcohol and cocamide MIPA is stable over time, even at low temperatures (<0° C.) and, mixed with an activator without ethoxylated emulsifiers, provides a transparent mixture that gives a homogeneous and long-lasting intense colour result without the need to add direct colourants. Said composition constitutes a further object of the invention.

In the context of the present invention, the term emulsifier means both an emulsifier and a surfactant. Non-ethoxylated emulsifier means an emulsifier substantially free of ethoxylated compounds except for those incidentally present as minor components, less than 0.5%, more preferably less than 0.1%, with respect to the total weight of the composition “Activator” means an agent capable of promoting the oxidative and coupling reaction between primary colourants and couplers. Examples of activators include hydrogen peroxide, carbamide peroxide, perborates and persulphates or peracids, preferably hydrogen peroxide.

The activator can be mixed with the hair dye composition in ratios generally from 1:1 to 1:2, depending on the desired result. A lower amount of activator promotes the concentration of colourants and is useful when the purpose is to camouflage grey hair; a higher amount of activator is useful when seeking to tone after bleaching or provide on-trend tones.

The composition according to the invention has pH 6.5-7.5, preferably 7, and after being mixed with the activator (pH of the mixture 6-7) is left applied for 2 to 60 minutes at room temperature and then rinsed.

The polyglycerol esters are chosen for example from: polyglyceryl-6-laurate (SABO), polyglyceryl-4-laurate (SABO), polyglyceryl-3-diisosterate (BASF), polyglyceryl-10-sterate (BASF), polyglyceryl-10-laurate (SD chemicals), polyglyceryl-4-caprate (SD chemicals), olive oil polyglyceryl-6-esters (SABO). Preferably the polyglycerol ester is polyglyceryl-6-laurate and/or polyglyceryl-3-diisosterate.

The polyglycerol esters are present in percentages by weight on the total weight of the composition from 3 to 20%, preferably from 5 to 15%; the oleic acid is present in percentages by weight on the total weight of the composition from 3 to 15%; the oleic alcohol is present in percentages by weight on the total weight of the composition from 0.5 to 10% and the cocamide MIPA is present in percentages by weight on the total weight of the composition from 0.5 to 10%.

The composition of the invention may contain other non-ethoxylated emulsifiers preferably selected from olefin sulfonates, taurate, alaninates, glutamates, isethionates, sulfosuccinates, betaines, glucosides. Sulfate surfactants, such as SLS and SLES, known for their irritant potential on the skin, are preferably excluded.

As consistency factors, the composition of the invention may contain fatty alcohols, preferably C12-C14, for example lauryl alcohol and myristyl alcohol.

To adjust the pH, the composition may contain, in percentages between 0.01 and 2%: arginine, lysine, monoisopropanolamine (MIPA), aminopropanol (2AP), dimethylglucamine (DMG), aminomethylpropanol (AMP), sodium hydroxide, potassium hydroxide, lactic acid, citric acid, phosphoric acid, phosphonic acid. The composition preferably contains no ethanolamine (MEA).

Examples of diluents comprise water, low molecular weight aliphatic mono- or poly-alcohols, their esters and ethers, for example aliphatic alcohols, in particular from 1 to 4 carbon atoms, such as ethanol, n-propanol, isopropanol, butanol, isobutanol; bivalent or trivalent alcohols, in particular having from 2 to 6 carbon atoms such as 1,3-propanediol, 1,4-butanediol, 1,5-pentanediol, 1,6-hexanediol, 1,2,6-hexantriol, glycerin, butylene glycol, dimethyl-isosorbide, propylene carbonate

Examples of non-silicone conditioners comprise cetrimonium chloride, soytrimonium chloride, lauryl/myristyl polyricinoleate, polyglyceryl-3 betainate acetate, polyglyceryl-3 betainate citrate, isoamyl laurate, myristyl lactate, panthenol, vitamins and pro-vitamins, sugars, vegetable oils such as meadowfoam seed oil, hydrogenated ethylexyl olivate & hydrogenated olive oil unsaponifiables.

Oxidation dyes are preferably chosen from 1-Acetoxy-2-Methylnaphthalene, 5-Amino-4-Chloro-o-Cresol, 4-Amino-m-Cresol, 3-Amino-2,4-Dichlorophenol, 3-Amino-2,4-Dichlorophenol, 2-Amino-5-Ethylphenol, 2-Amino-4-Hydroxyethylaminoanisole, 2-Amino-4-Hydroxyethylaminoanisole, 2-Amino-3-Hydroxypyridine, 4-Amino-2-Hydroxytoluene, m-Aminophenol, p-Aminophenol, 1,3-Bis-(2,4-Diaminophenoxy)propane, 2,6-Bis(2-Hydroxyethoxy)-3,5-Pyridinediamine, N,N-Bis(2-Hydroxyethyl)-p-Phenylenediamine, 4-Chlororesorcinol, 2,4-Diaminophenoxyethanol, 2,6-Diaminopyridine, 2,6-Dihydroxy-3,4-Dimethylpyridine, 2,6-Dihydroxyethylaminotoluene, Dihydroxyindole, 5,6-Dihydroxyindoline, 2,6-Dimethoxy-3,5-Pyridinediamine, 1-Hexyl-4,5-Diamino Pyrazole Sulfate, Hydroxyethyl-3,4-Methylenedioxyaniline, Hydroxyethyl-p-Phenylenediamine, 6-Hydroxyindole, 6-Methoxy-2-methylamino-3-aminopyridine, 2-Methoxymethyl-p-Aminophenol, 2-Methoxymethyl-p-Phenylenediamine, 2-Methoxy-p-Phenylenediamine, p-Methylaminophenol, 2-Methyl-5-Hydroxyethylaminophenol, 2-Methyl-1-Naphthol, 2-Methylresorcinol, 1,5-Naphthalenediol, 1,7-Naphthalenediol, 2,3-Naphthalenediol, 2,7-Naphthalenediol, 1-Naphthol, Phenyl Methyl Pyrazolone, resorcinol, tetraaminopyrimidine, toluene-2,5-diamine, p-phenylenediamine or their salts.

The composition of the invention preferably does not contain direct colourants and resorcinol and its derivatives.

The composition of the invention may contain auxiliary agents such as chelating agents, antioxidants or reducing agents, preservatives, fragrance solubilizers and other ingredients commonly used in hair care compositions.

The invention will be described in detail in the following examples, in which the components are named by the INCI nomenclature (Decision of the European Community 2006/257/EC and subsequent amendments—International nomenclature cosmetic ingredient).

Inventive and comparative formulations used in the examples below are disclosed in table 2. The formulas contain ethanolamine (MEA) as an alkalinizer to be able to compare them with market references characterized by the presence of this alkalinizer.

TABLE 2
Nuances 5.3 according to the invention and comparative

| | A1 | A2 | A3 | A4* | A5 | A6* |
|---|---|---|---|---|---|---|
| AQUA | as needed | as needed | as needed | as needed | as needed | as needed |
| PROPYLENE GLYCOL | 9.5 | 9.5 | 9.5 | 9.5 | 9.5 | 9.5 |
| COCAMIDE MIPA | 0 | 12 | 0 | 5.5 | 0 | 0 |
| OLEYL ALCOHOL | 0 | 0 | 12 | 6.5 | 0 | 0 |
| OLEIC ACID | 0 | 0 | 0 | 0 | 0 | 12 |
| LAURYL ALCOHOL | 1.05 | 1.05 | 1.05 | 1.05 | 1.05 | 1.05 |
| MYRISTYL ALCOHOL | 0.45 | 0.45 | 0.45 | 0.45 | 0.45 | 0.45 |
| ISOPROPYL ALCOHOL | 15 | 15 | 15 | 15 | 15 | 15 |
| SODIUM C14-16 OLEFINSULFONATE | 10 | 10 | 10 | 10 | 10 | 10 |
| POLYGLYCERYL-6 LAURATE | 10 | 10 | 10 | 10 | 0 | 0 |
| POLYGLYCERYL-3 DIISOSTEARATE | 0 | 0 | 0 | 0 | 10 | 10 |
| ETHANOLAMINE | 1 | 1 | 1 | 1 | 1 | 1 |
| P-PHENYLENEDIAMINE | 0.1 | 0.1 | 0.1 | 0.1 | 0.1 | 0.1 |
| p-AMINOPHENOL | 0.55 | 0.55 | 0.55 | 0.55 | 0.55 | 0.55 |
| HYDROXYETHYL-3,4- | 1.38 | 1.38 | 1.38 | 1.38 | 1.38 | 1.38 |
| METHYLENEDIOXYANILINE HCL | | | | | | |
| 2,6-DIHYDROXY-3,4- | 0.35 | 0.35 | 0.35 | 0.35 | 0.35 | 0.35 |
| DIMETHYLPYRIDINE | | | | | | |
| 1-HYDROXYETHYL-4,5-DIAMINO | 0.7 | 0.7 | 0.7 | 0.7 | 0.7 | 0.7 |
| PYRAZOLE SULFATE | | | | | | |
| LACTIC ACID | as needed | as needed | as needed | as needed | as needed | as needed |
| | to pH 7 | to pH 7 | to pH 7 | to pH 7 | to pH 7 | to pH 7 |
| SODIUM SULFITE | 0.5 | 0.5 | 0.5 | 0.5 | 0.5 | 0.5 |
| EDTA | 0.2 | 0.2 | 0.2 | 0.2 | 0.2 | 0.2 |
| ERYTHORBIC ACID | 0.4 | 0.4 | 0.4 | 0.4 | 0.4 | 0.4 |
| PARFUM (FRAGRANCE) | 0.5 | 0.5 | 0.5 | 0.5 | 0.5 | 0.5 |
| pH | 7.06 | 7.06 | 7.06 | 7.03 | 7.06 | 7.07 |

The asterisk indicates the compositions according to the invention (F4 and F6).

TABLE 3
ethoxylate-free liquid activator

| | B1* |
|---|---|
| AQUA | as needed |
| HYDROGEN PEROXIDE | 1.5 |
| SODIUM HYDROXIDE | 0.009 |
| DISODIUM | 0.05 |
| PYROPHOSPHATE | |
| SODIUM STANNATE | 0.1 |
| ETIDRONIC ACID | 0.16 |

In the following tests, market products will also be used as comparative formulas, characterized as follows:
reference 1: liquid demi-permanent colour without ethoxylates at pH 6.5 to be mixed with a suitable activator containing ethoxylates (1:1 ratio), to give a mixture at pH 6.
reference 2: liquid demi-permanent colour with ethoxylates at pH 10 (alkalizing agent MEA) to be mixed with a suitable activator without ethoxylates (1:1 ratio), to give a mixture at pH 6.7.
reference 3: liquid demi-permanent colour with ethoxylates at pH 10 (alkalizing MEA) to be mixed with a special activator with ethoxylates (1:1 ratio), to give a mixture at pH 9.5.

Further examples of formulations of the invention are given in Table 4

| | A8* | A9* | A10* | A11* | A12* | A13* | A14* |
|---|---|---|---|---|---|---|---|
| AQUA | as needed | as needed | as needed | as needed | as needed | as needed | as needed |
| PROPYLENE GLYCOL | 9.5 | 9.5 | 9.5 | 9.5 | 9.5 | 9.5 | 9.5 |
| OLEYL ALCOHOL | 6 | 6 | 6 | 0.5 | 6 | 6 | 10 |
| OLEIC ACID | 0 | 0 | 0 | 0 | 0 | 0 | 0 |
| LAURYL ALCOHOL | 1.05 | 1.05 | 1.05 | 1.05 | 1.05 | 1.05 | 1.05 |
| MYRISTYL ALCOHOL | 0.45 | 0.45 | 0.45 | 0.45 | 0.4 | 0.45 | 0.45 |
| ISOAMYL LAURATE | 0.5 | 0.5 | 0.5 | 0.2 | 0.2 | 0 | 0 |
| MEADOWFOAM SEED OIL | 0 | 0 | 0 | 0 | 0 | 1 | 1 |
| COCAMIDE MIPA | 5.1 | 5.1 | 5.1 | 10 | 5.1 | 5.1 | 0.5 |
| ISOPROPYL ALCOHOL | 15 | 15 | 15 | 15 | 15 | 15 | 15 |
| SODIUM C14-16 | 4 | 0 | 0 | 0 | 0 | 0 | 0 |
| OLEFINSULFONATE | | | | | | | |
| SODIUM METHYL COCOYL | 0 | 4 | 0 | 0 | 0 | 0 | 0 |
| TAURATE | | | | | | | |
| SODIUM MYRISTOYL | 0 | 0 | 0 | 4 | 0 | 0 | 0 |
| GLUTAMATE | | | | | | | |
| SODIUM LAUROYL METHYL | 0 | 0 | 4 | 0 | 0 | 0 | 0 |
| ISETHIONATE | | | | | | | |
| SODIUM COCOYL | 0 | 0 | 0 | 0 | 0 | 0 | 0 |
| ISETHIONATE | | | | | | | |
| POLYGLYCERYL-6 LAURATE | 10 | 10 | 10 | 10 | 0 | 0 | 0 |
| POLYGLYCERYL-3 | 0 | 0 | 0 | 0 | 0 | 0 | 0 |
| DIISOSTEARATE | | | | | | | |
| POLYGLYCERYL-4 LAURATE | 0 | 0 | 0 | 0 | 15 | 0 | 0 |
| POLYGLYCERYL-4 CAPRATE | 0 | 0 | 0 | 0 | 0 | 10 | 0 |
| OLIVE OIL POLYGLYCERYL- | 0 | 0 | 0 | 0 | 0 | 0 | 5 |
| 6-ESTERS | | | | | | | |
| POLYGLYCERYL-2-OLEATE | 0 | 0 | 0 | 0 | 0 | 0 | 0 |
| POLYGLYCERYL-10-STEARATE | 0 | 0 | 0 | 0 | 0 | 0 | 0 |
| SWEET ALMOND OIL | 0 | 0 | 0 | 0 | 0 | 0 | 0 |
| POLYGLYCERYOL-6-ESTERS | | | | | | | |
| POLYGLYCERYL-6-STEARATE | 0 | 0 | 0 | 0 | 0 | 0 | 0 |
| POTASSIUM HYDROXIDE | 1.15 | 1.15 | 1.15 | 1.15 | 1.15 | 1.15 | 1.15 |
| DIMETHYLGLUCAMINE | 0 | 0 | 0 | 0 | 0 | 0 | 0 |
| 1-AMINO-2-PROPANOL | 0 | 0 | 0 | 0 | 0 | 0 | 0 |
| LACTIC ACID | as needed | as needed | as needed | as needed | as needed | as needed | as needed |
| | to pH 7 | to pH 7 | to pH 7 | to pH 7 | to pH 7 | to pH 7 | to pH 7 |
| FOSFONIC ACID | 0 | 0 | 0 | 0 | 0 | 0 | 0 |
| SODIUM SULFITE | 0.5 | 0.5 | 0.5 | 0.5 | 0.5 | 0.5 | 0.5 |
| EDTA | 0.2 | 0.2 | 0.2 | 0.2 | 0.2 | 0.2 | 0.2 |
| ERYTHORBIC ACID | 0.4 | 0.4 | 0.4 | 0.4 | 0.4 | 0.4 | 0.4 |
| P-PHENYLENEDIAMINE | 0.91 | 0.91 | 0.91 | 0.91 | 0.91 | 0.91 | 0.91 |
| 2-AMINO-3-HYDROXYPIRIDINE | 0.12 | 0.12 | 0.12 | 0.12 | 0.12 | 0.12 | 0.12 |
| HYDROXYETHYL-3,4- | 1.2 | 1.2 | 1.2 | 1.2 | 1.2 | 1.2 | 1.2 |
| METHYLENEDIOXY ANILINE | | | | | | | |
| HCL | | | | | | | |
| m-AMINOPHENOL | 0.15 | 0.15 | 0.15 | 0.15 | 0.15 | 0.15 | 0.15 |
| 2,4-DIAMINOPHENOXYETHANOL | 0.1 | 0.1 | 0.1 | 0.1 | 0.1 | 0.1 | 0.1 |
| HCL | | | | | | | |
| PARFUM (FRAGRANCE) | 0.5 | 0.5 | 0.5 | 0.5 | 0.5 | 0.5 | 0.5 |

| | A15* | A16* | A17* | A18* | A19* | A20* |
|---|---|---|---|---|---|---|
| AQUA | as needed | as needed | as needed | as needed | as needed | as needed |
| PROPYLENE GLYCOL | 9.5 | 9.5 | 9.5 | 9.5 | 9.5 | 9.5 |
| OLEYL ALCOHOL | 6 | 6 | 6 | 6 | 0 | 0 |
| OLEIC ACID | 0 | 0 | 0 | 0 | 12 | 3 |
| LAURYL ALCOHOL | 1.05 | 1.05 | 1.05 | 1.05 | 1.05 | 1.05 |
| MYRISTYL ALCOHOL | 0.45 | 0.45 | 0.45 | 0.45 | 0.45 | 0.45 |
| ISOAMYL LAURATE | 0 | 0 | 0 | 0 | 0.5 | 0 |
| MEADOWFOAM SEED OIL | 1 | 0.5 | 1 | 1 | 0 | 1 |
| COCAMIDE MIPA | 5.1 | 5.1 | 5.1 | 5.1 | 0 | 0 |
| ISOPROPYL ALCOHOL | 15 | 15 | 15 | 15 | 15 | 15 |
| SODIUM C14-16 | 0 | 0 | 0 | 0 | 0 | 0 |
| OLEFINSULFONATE | | | | | | |
| SODIUM METHYL COCOYL | 0 | 0 | 0 | 0 | 0 | 0 |
| TAURATE | | | | | | |
| SODIUM MYRISTOYL | 0 | 0 | 0 | 0 | 0 | 0 |
| GLUTAMATE | | | | | | |
| SODIUM LAUROYL METHYL | 0 | 0 | 0 | 0 | 0 | 0 |
| ISETHIONATE | | | | | | |
| SODIUM COCOYL | 0 | 0 | 0 | 0 | 4 | 4 |
| ISETHIONATE | | | | | | |
| POLYGLYCERYL-6 LAURATE | 0 | 0 | 0 | 0 | 0 | 0 |
| POLYGLYCERYL-3 | 0 | 0 | 0 | 0 | 10 | 10 |
| DIISOSTEARATE | | | | | | |
| POLYGLYCERYL-4 LAURATE | 0 | 0 | 0 | 0 | 0 | 0 |
| POLYGLYCERYL-4 CAPRATE | 0 | 0 | 0 | 0 | 0 | 0 |
| OLIVE OIL POLYGLYCERYL- | 0 | 0 | 0 | 0 | 0 | 0 |
| 6-ESTERS | | | | | | |
| POLYGLYCERYL-2-OLEATE | 20 | 0 | 0 | 0 | 0 | 0 |
| POLYGLYCERYL-10-STEARATE | 0 | 10 | 0 | 0 | 0 | 0 |
| SWEET ALMOND OIL | 0 | 0 | 10 | 0 | 0 | 0 |
| POLYGLYCERYOL-6-ESTERS | | | | | | |
| POLYGLYCERYL-6-STEARATE | 0 | 0 | 0 | 10 | 0 | 0 |
| POTASSIUM HYDROXIDE | 1.15 | 1.15 | 1.15 | 0 | 0 | 0 |
| DIMETHYLGLUCAMINE | 0 | 0 | 0 | 0 | 2.5 | 2.5 |
| 1-AMINO-2-PROPANOL | 0 | 0 | 0 | 0.7 | 0 | 0 |
| LACTIC ACID | as needed | 0 | as needed | as needed | as needed | as needed |
| | to pH 7 | | to pH 7 | to pH 7 | to pH 7 | to pH 7 |
| FOSFONIC ACID | 0 | as needed | 0 | 0 | 0 | 0 |
| | | to pH 7 | | | | |
| SODIUM SULFITE | 0.5 | 0.5 | 0.5 | 0.5 | 0.5 | 0.5 |
| EDTA | 0.2 | 0.2 | 0.2 | 0.2 | 0.2 | 0.2 |
| ERYTHORBIC ACID | 0.4 | 0.4 | 0.4 | 0.4 | 0.4 | 0.4 |
| P-PHENYLENEDIAMINE | 0.91 | 0.91 | 0.91 | 0.91 | 0.91 | 0.91 |
| 2-AMINO-3-HYDROXYPIRIDINE | 0.12 | 0.12 | 0.12 | 0.12 | 0.12 | 0.12 |
| HYDROXYETHYL-3,4- | 1.2 | 1.2 | 1.2 | 1.2 | 1.2 | 1.2 |
| METHYLENEDIOXY ANILINE | | | | | | |
| HCL | | | | | | |
| m-AMINOPHENOL | 0.15 | 0.15 | 0.15 | 0.15 | 0.15 | 0.15 |
| 2,4-DIAMINOPHENOXYETHANOL | 0.1 | 0.1 | 0.1 | 0.1 | 0.1 | 0.1 |
| HCL | | | | | | |
| PARFUM (FRAGRANCE) | 0.5 | 0.5 | 0.5 | 0.5 | 0.5 | 0.5 |

### EXAMPLE 1: SLIP TEST

This test aims to evaluate the tendency of a hair product to slip or not from the head after application.

A plastic support positioned at an angle of 300 has been prepared. One gram for each of the compositions of Table 2 was mixed in 1:1 ratio with activator B1* and positioned on top of the inclined plane. The behaviour of the various products was observed for 1 minute by measuring the distance travelled after 10, 20, 30 and 60 seconds (s).

The data are shown in Table 5 in centimetres (cm) with respect to time in seconds.

TABLE 5
slip test with inclined plane

| 1:1 | | | | |
|---|---|---|---|---|
| Mix Formulas | 10 s | 20 s | 30 s | 60 s |
| A1 + B1* | 2 | 5 | 8 | 15 |
| A2 + B1* | 0.5 | 1 | 2 | 4 |
| A3 + B1* | 1 | 2 | 4 | 7 |
| A4* + B1* | 0 | 0.5 | 0.5 | 1 |
| A5 + B1* | 1 | 3 | 6 | 10 |
| A6* + B1* | 0 | 0.5 | 0.5 | 1 |

From the data in Table 5, it can be seen how the formulas according to the invention have good endurance even when mixed with an activator without ethoxylates B1*. This shows that only the combinations of emulsifiers according to the invention produce suitable performance

### EXAMPLE 2: ACCELERATED STABILITY TEST

The tests, conducted according to standard ISO/TR18811 of February 2018, subject the formulas to extreme conditions for three months in order to predict and guarantee the stability thereof over time for the period of storage, transport and marketing of the product from 3 to 5 years.

The formulations in Table 2 are stored in closed glass containers at various temperatures for different periods of time. The chemical-physical parameters are evaluated at the times indicated both instrumentally (pH, viscosity, etc.) and visually in relation to a freshly prepared reference standard. The same procedure is applied to two acidic demi-liquid dyes on the market, one with and one without ethoxylated emulsifiers.

Table 6 shows the visual parameter of the separation/precipitation evaluated by three expert panellists.

TABLE 6
dye stability analysis

| | Room temperature | 50° C. for 15 | 40° C. for 90 | 4° C. for 90 | −10° C. for 2 | −2° C. for 15 |
|---|---|---|---|---|---|---|
| Formula | for 90 days | days | days | days | days | days |
| A4* | No separation | No separation | No separation | No separation | No separation | No separation |
| A6* | No separation | No separation | No separation | No separation | No separation | No separation |
| Reference 1 | No separation | No separation | No separation | No separation | No separation | Separation |
| Reference 2 | No separation | No separation | No separation | No separation | No separation | No separation |

The data reported in Table 6 demonstrate that the formulations of the invention are more stable at temperature <0° C. with respect to the comparative formulations without ethoxylates with analogous pH, and comparable to those with ethoxylates at alkaline pH.

### EXAMPLE 3: TRANSPARENCY PANEL TEST

The test was conducted by evaluating the mixtures of the various dyes with the appropriate activators (in a 1:1 ratio) by 10 expert panellists.

The mixtures were prepared in small colourless glass beakers and any bubbles were removed by sonicator.

Behind each beaker, a card bearing a letter was placed in an upright position and each individual was asked to stand in front of the beaker and observe through it, evaluating the sharpness with which the letter appeared, thus making a ranking of the formulations from the most opaque to the most transparent.

The results are shown in Table 7.

TABLE 7
transparency panel test

| | | | Somewhat | Trans- |
|---|---|---|---|---|
| panellist | opaque | Slightly opaque | transparent | parent |
| 1 | Reference 1 + | Reference 3 + | Reference 2 + | A4* + |
| | specific | specific activator | specific activator | B1* |
| | activator | | | |
| 2 | Reference 1 + | Reference 2 | Reference 3 + | A4* + |
| | specific | | specific activator | B1* |
| | activator | | | |
| 3 | Reference 1 + | Reference 3 + | Reference 2 + | A4* + |
| | specific | specific activator | specific activator | B1* |
| | activator | | | |
| 4 | Reference 1 + | Reference 3 + | Reference 2 + | A4* + |
| | specific | specific activator | specific activator | B1* |
| | activator | | | |
| 5 | Reference 1 + | Reference 3 + | Reference 2 + | A4* + |
| | specific | specific activator | specific activator | B1* |
| | activator | | | |
| 6 | Reference 3 | Reference 1 | Reference 2 + | A4* + |
| | | | specific activator | B1* |
| 7 | Reference 1 + | Reference 3 + | Reference 2 + | A4* + |
| | specific | specific activator | specific activator | B1* |
| | activator | | | |
| 8 | Reference 1 + | Reference 3 + | Reference 2 + | A4* + |
| | specific | specific activator | specific activator | B1* |
| | activator | | | |
| 9 | Reference 1 + | Reference 3 + | Reference 2 + | A4* + |
| | specific | specific activator | specific activator | B1* |
| | activator | | | |
| 10 | Reference 1 + | Reference 3 + | Reference 2 + | A4* + |
| | specific | specific activator | specific activator | B1* |
| | activator | | | |

From the data in Table 7, it can be seen that the ethoxylate-free formula according to the invention is the best in terms of transparency, also mixed with ethoxylate-free activator B1*.

### EXAMPLE 4: INSTRUMENTAL TRANSPARENCY TEST

To carry out this test, it was decided to use the LUMiSizer, a tool designed to analyse the distribution of particles in aqueous, non-aqueous, Newtonian or non-Newtonian systems.

The instrument is able to allocate 12 cuvettes in which the samples to be analysed are inserted, it is equipped with a centrifuge that we used at 3000 RPM for 10 seconds to homogenise the samples, eliminating air bubbles. The instrument evaluates the sample through a transmittance measurement carried out along the entire cuvette (2 mm) and expressed as an average of the values measured at 9 points. The wavelength used is in the near infrared (865 nm). This wavelength guarantees to evaluate the transmittance excluding the interference of the colourants present in the dyes. The analysis is carried out at 25° C. using water as a reference standard.

The results obtained are reported in Table 8, expressed as a percentage of the ray that crosses the formulation with respect to the incident ray. The higher the percentage, the more transparent the formula.

TABLE 8
LUMiSizer transmittance

| | Average |
|---|---|
| Mix Formulas | % |
| A4* + B1* | 51.21 |
| Reference 1 + specific | 21.10 |
| activator | |
| Reference 2 + specific | 49.83 |
| activator | |
| Reference 3 + specific | 45.27 |
| activator | |

The instrumental data confirms that of the panel test OF EXAMPLE 3: the formula according to the invention is the most transparent. Considering the results of panel testing and transmittance, references 2 and 3 are substantially comparable

### EXAMPLE 5: WASHING COLOR FASTNESS TEST

The colour fastness on hair is affected by fibre damage. The more damaged and porous the hair, the worse the colour fastness to washing.

To simulate hair damage, very light blonde IHIP level 10 strands were bleached with 9-level bleaching powder mixed with 30 vol activator in a 1:2 ratio, applied to the strands and left on for 40 minutes at 30° C. The strands were then rinsed, shampooed and dried for 30 minutes at 55° C. in a special dryer. Subsequently, the strands were dyed using formulas A4* mixed in a 1:1 ratio with activator B1* and reference 3 with specific activator mixed according to the instructions. The colour was left on the strands for 20 minutes at a temperature of 30° C. The strands were then rinsed and dried for 30 minutes at 55° C. in a special dryer. The strands are then rinsed with tap water at 37±2° C. and a flow rate of 4±0.1 L/min. Using a precision Handy Step S dispenser, 1 ml of shampoo is applied to each strand and evenly distributed over the entire length. The strands are rinsed with running tap water (same conditions as above) and then dried in a dryer for 20 minutes at 55° C. These steps are repeated for each wash.

A Konica Minolta colorimeter, equipped with SpectraMagic NX Software, is used to evaluate the fastness of the colour to washing.

In the CIELAB colour space, L* indicates the brightness while a* and b* are the chromaticity coordinates. a* and b* indicate the colour directions+a* is the red direction, −a* is the green direction, +b* is the yellow direction and −b* is the blue direction. The colour differences can be expressed by the values ΔE, which are defined by the following equation:

$$Δ E = [ ( Δ {L}^{*} ) 2 + ( Δ {a}^{*} ) 2 + ( Δ {b}^{*} ) 2 ] 1 / 2$$

Measurement is performed at time zero and repeated after 3, 6, 9 and 12 shampoo washes. The colour differences can be expressed by the values ΔE: the smaller the value of Δ and the less the colour loss after washing. Table 8 shows the ΔE data in percentage, obtained after 12 washes.

TABLE 9
Colour loss after 12 washes

| Formula | ΔE |
|---|---|
| A4* + B1* | 15% |
| Reference 3 + relative activator | 14% |

From the data in Table 9 it can be seen that the ethoxylate-free colouration according to the invention is comparable in terms of colour loss to the reference dye, which is only partially ethoxylate-free.

1. A method for demi-permanent hair colouring which comprises
applying a transparent mixture to hair consisting of:
a) a first composition in liquid form free of ethoxylated emulsifiers and polyethylene glycols comprising oxidative dyes and a combination of polyglyceryl esters with oleic acid or a combination of polyglycerol esters with oleic alcohol and cocamide MIPA; wherein the polyglycerol esters are present in percentages by weight on the total weight of the composition from 3 to 20%, the oleic acid is present in percentages by weight on the total weight of the composition from 3 to 15%, the oleic alcohol is present in percentages by weight on the total weight of the composition from 0.5 to 10% and the cocamide MIPA is present in percentages by weight on the total weight of the composition from 0.5 to 10%.
b) a second ethoxylate-free formulation comprising an activator.

2. The method according to claim 1, wherein the compositions a) and b) are mixed in a weight ratio of 1:1 to 1:2

3. The method according to claim 1, wherein the compositions a) and b) are free of silicones and acrylic polymers.

4. The method according to claim 1, wherein the composition a) has a pH between 6.5 and 7.5.

5. The method according to claim 1, wherein the composition a) is stable over time, even at temperatures <0° C.

6. The method according to claim 1, wherein the polyglycerol esters are selected from: polyglyceryl-6-laurate, polyglyceryl-4-laurate, polyglyceryl-3-diisosterate, polyglyceryl-10-sterate, polyglyceryl-10-laurate, polyglyceryl-4-caprate, olive oil and polyglyceryl-6-esters.

7. The method according to claim 1, wherein the oxidative dyes are selected from 1-Acetoxy-2-Methylnaphthalene, 5-Amino-4-Chloro-o-Cresol, 4-Amino-m-Cresol, 3-Amino-2,4-Dichlorophenol, 3-Amino-2,4-Dichlorophenol, 2-Amino-5-Ethylphenol, 2-Amino-4-Hydroxyethylaminoanisole, 2-Amino-4-Hydroxyethylaminoanisole, 2-Amino-3-hydroxypyridine, 4-Amino-2-hydroxytoluene, m-Aminophenol, p-Aminophenol, 1,3-Bis-(2,4-Diaminophenoxy)propane, 2,6-Bis(2-Hydroxyethoxy)-3,5-Pyridinediamines, N,N-Bis(2-Hydroxyethyl)-p-Phenylenediamine, 2,4-Diaminophenoxyethanol, 2,6-Diaminopyridine, 2,6-Dihydroxy-3,4-Dimethylpyridine, 2,6-Dihydroxyethylaminotoluene, Dihydroxyindole, 5,6-Dihydroxyindoline, 2,6-Dimethoxy-3,5-Pyridinediamine, 1-Hexyl 4,5-Diamino Pyrazole Sulfate, Hydroxyethyl-3,4-Methylenedioxyaniline, Hydroxyethyl-p-Phenylenediamine, 6-Hydroxyindole, 6-Methoxy-2-methylamino-3-aminopyridine, 2-Methoxymethyl-p-Aminophenol, 2-Methoxymethyl-p-Phenylenediamine, 2-Methoxy-p-Phenylenediamine, p-Methylaminophenol, 2-Methyl-5-Hydroxyethylaminophenol, 2-Methyl-1-Naphthol, 1,5-Naphthalenediol, 1,7-Naphthalenediol, 2,3-Naphthalenediol, 2,7-Naphthalenediol, 1-Naphthol, Phenyl Methyl Pyrazolone, resorcinol, tetraaminopyrimidine, toluene-2,5-diamine, p-phenylenediamine.

8. The method according to claim 7, wherein the oxidative dyes do not comprise resorcinol and its derivatives.

9. The method according to claim 1, wherein the first composition comprises non-ethoxylated surfactants/emulsifiers chosen from olefin sulfonates, taurates, glutamates, alaninates, isethionates, sulfosuccinates, betaines, glucosides.

10. The method according to claim 1, wherein the first composition does not comprise sulphate surfactants.

11. The method according to claim 1, wherein the first composition comprises C₁₂-C₁₄ fatty alcohols, pH adjusting agents excluding ethanolamine, diluents, non-silicone conditioners, chelating agents and/or reducing agents, preservatives, PEG-free solubilizers for fragrances.

12. The method according to claim 1, wherein the activator comprises hydrogen peroxide.

13. A liquid compositions at pH 6.5-7.5 free of ethoxylated emulsifiers and polyethylene glycols comprising oxidative dyes and a mixture of polyglyceryl esters with oleic acid or a mixture of polyglyceryl esters with oleyl alcohol and cocamide MIPA.

14. The method according to claim 1, wherein the polyglycerol esters are polyglyceryl-6-laurate and/or polyglyceryl-3-diisosterate.
